(19) United States
(12) Patent Application Publication
(10) Pub. No.: US 2023/0192631 A1
(43) Pub. Date: Jun. 22, 2023

(54) Biphenyl Derivatives

(71) Applicant: Hoffmann-La Roche Inc., Little Falls, NJ (US)

(72) Inventors: Stefan BERCHTOLD, Binningen (CH); Guido GALLEY, Rheinfelden (DE); Katrin GROEBKE ZBINDEN, Liestal (CH); Wolfgang GUBA, Muellheim (DE); Daniel HUNZIKER, Moehlin (CH); Danny KRUMM, Basel (CH)

(73) Assignee: Hoffmann-La Roche Inc., Little Falls, NJ (US)

(21) Appl. No.: 17/996,325

(22) PCT Filed: Apr. 14, 2021
(86) PCT No.: PCT/EP2021/059599

§ 371 Date: Oct. 14, 2022

(30) Foreign Application Priority Data
Apr. 16, 2020 (EP) 20169766.1

(51) Int. Cl. C07D 249/08; C07D 239/36; C07D 295/145; C07C 233/55; C07D 233/60; C07C 65/21

(52) U.S. Cl. CPC C07D 249/08; C07D 239/36; C07D 295/145; C07C 233/55; C07D 233/60; C07C 65/21

(57) ABSTRACT

The invention relates to a compound of formula (I) wherein R¹—R³ are as defined in the description and in the claims. The compound of formula (I) can be used as a medicament.

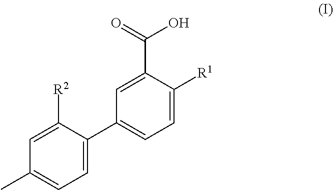

The present invention relates to organic compounds useful for therapy and/or prophylaxis in a mammal, and in particular to compounds that modulate cGAS activity.

The invention relates in particular to a compound of formula (I)

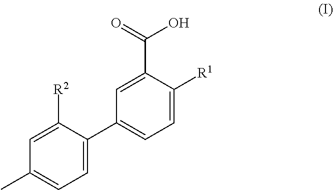

wherein
R¹ is —NHC(O)—R³ or —OR³;
R² is halogen; and
R³ is triazolylalkyl, alkyl(oxo-dihydropyrimidinyl)alkyl, morpholinylalkyl, imidazolylalkyl, (dioxo-thiazinanyl)alkyl or phenylalkyl;
or a pharmaceutically acceptable salt or ester thereof.

Cytokines are responsible for modulation of the innate immune response and the dysregulation of pro-inflammatory cytokines has been associated with severe systemic inflammation and autoimmune diseases, many of which lack efficient therapy as of today.

Vertebrates possess an innate and adaptive immune system as protection against pathogens and other challenges. The innate immune system is an evolutionary old system that is present beyond vertebrates. Unlike the adaptive immune system, it does not require priming or training, but works as a general physical barrier (e.g. skin) or by detection of specific patterns. One universal pattern to trigger the innate immune system is the detection of cytosolic double stranded DNA, which leads to Type I Interferon response. Sources of cytosolic dsDNA could be from bacterial or viral infection but as well accumulated self-DNA.

The cytosolic enzyme cyclic GMP-AMP Synthase (cGAS) is a sensor for cytosolic double stranded DNA. Binding of dsDNA results in the generation of the cyclic di-nucleotide 2,3-cGAMP by enzymatic linkage of ATP and GTP. 2,3-cGAMP acts as secondary messenger and binds to the Stimulator of Interferon Genes (STING), which resides in the endoplasmatic reticulum. Upon binding of 2,3-cGAMP, STING translocates to the perinuclear Golgi, where it associates with the TANK binding kinase 1 (TBK1) and recruits and phosphorylates Interferon Response Factor 3 (IRF3). Ultimately this results in the production of Type I Interferon (I IFN), other cytokines like IL-6, TNFα, IL1β and chemokines—essential factors for host defense against invading pathogens. However, inappropriate or chronic production of type I IFN and other pro-inflammatory cytokines are associated with severe systemic inflammation and autoimmune diseases. For instance, IFN signaling is involved in SLE, cutaneous skin diseases (dermatomyositis, and cutaneous lupus), interstitial pulmonary fibrosis, Sjogren syndrome, and type I diabetes (G. Trinchieri, J Exp Med. 2010 207(10): 2053-63). Other pro-inflammatory cytokine such as TNFα and IL1β play an important role in inflammatory bowel disease, NASH, juvenile inflammatory arthritis, ankylosing spondylitis and gout.

Chronic activation of cGAS/STING causes severe systemic inflammation. Evidence for its role in inflammation in the clinic comes from monogenic diseases. Patients with deficiencies in nucleic acid modifying enzymes, like Trex1, RNaseH2 and SAMHD1, suffer from Aicardi-Goutieres syndrome (AGS). The involvement of cGAS/STING was supported in Trex1 deficient mice that serve as a model for AGS.

Inhibition of the cGAS pathway which is upstream from the disease mediating cytokines is therefore a novel strategy in treating patients from multiple autoimmune diseases. Indications could include those linked to IFN signaling or those driven by TNFα and IL1β.

As of today many diseases caused by dysregulation of the innate immune system lack efficient therapies.

The compound of the invention binds to and modulates cGAS activity.

The compound of formula (I) is particularly useful in the treatment or prophylaxis of e.g. systemic lupus erythrematosus (SLE), cutaneous skin diseases like dermatomyositis or cutaneous lupus, interstitial pulmonary fibrosis, Sjogren syndrome, type I diabetes, inflammatory bowel disease, non-alcoholic steatohepatitis (NASH), juvenile inflammatory arthritis, ankylosing spondylitis, gout or Aicardi-Goutieres syndrome (AGS).

In the present description the term “alkyl”, alone or in combination, signifies a straight-chain or branched-chain alkyl group with 1 to 8 carbon atoms, particularly a straight or branched-chain alkyl group with 1 to 6 carbon atoms and more particularly a straight or branched-chain alkyl group with 1 to 4 carbon atoms. Examples of straight-chain and branched-chain C1-C8 alkyl groups are methyl, ethyl, propyl, isopropyl, butyl, isobutyl, tert.-butyl, sec.-butyl, the isomeric pentyls, the isomeric hexyls, the isomeric heptyls and the isomeric octyls, particularly methyl, ethyl, propyl, butyl and pentyl. Particular examples of alkyl are methyl, ethyl and propyl. Methyl and ethyl are particular examples of “alkyl” in the compound of formula (I).

The terms “halogen” or “halo”, alone or in combination, signifies fluorine, chlorine, bromine or iodine and particularly fluorine, chlorine or bromine, more particularly chlorine. The term “halo”, in combination with another group, denotes the substitution of said group with at least one halogen, particularly substituted with one to five halogens, particularly one to four halogens, i.e. one, two, three or four halogens.

The term “carbonyl”, alone or in combination, signifies the —C(O)— group.

The term “pharmaceutically acceptable salts” refers to those salts which retain the biological effectiveness and properties of the free bases or free acids, which are not biologically or otherwise undesirable. The salts are formed with inorganic acids such as hydrochloric acid, hydrobromic acid, sulfuric acid, nitric acid, phosphoric acid, particularly hydrochloric acid, and organic acids such as acetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid, N-acetylcystein. In addition these salts may be prepared form addition of an inorganic base or an organic base to the free acid. Salts derived from an inorganic base include, but are not limited to, the sodium, potassium, lithium, ammonium, calcium, magnesium salts. Salts derived from organic bases include, but are not limited to salts of primary, secondary, and tertiary amines, substituted amines including naturally occurring substituted amines, cyclic amines and basic ion exchange resins, such as isopropylamine, trimethylamine, diethylamine, triethylamine, tripropylamine, ethanolamine, lysine, arginine, N-ethylpiperidine, piperidine, polyamine resins. The compound of formula (I) can also be present in the form of zwitterions. Particularly preferred pharmaceutically acceptable salts of compounds of formula (I) are the salts of hydrochloric acid, hydrobromic acid, sulfuric acid, phosphoric acid, acetic acid, sodium and potassium.

The term “pharmaceutically acceptable esters” means that compounds of general formula (I) may be derivatised at functional groups to provide derivatives which are capable of conversion back to the parent compounds in vivo. Examples of such compounds include physiologically acceptable and metabolically labile ester derivatives, such as methoxymethyl esters, methylthiomethyl esters and pivaloyloxymethyl esters. Additionally, any physiologically acceptable equivalents of the compounds of general formula (I), similar to the metabolically labile esters, which are capable of producing the parent compounds of general formula (I) in vivo, are within the scope of this invention.

If one of the starting materials or compounds of formula (I) contain one or more functional groups which are not stable or are reactive under the reaction conditions of one or more reaction steps, appropriate protecting groups (as described e.g. in “Protective Groups in Organic Chemistry” by T. W. Greene and P. G. M. Wuts, 3^{rd} Ed., 1999, Wiley, N.Y.) can be introduced before the critical step applying methods well known in the art. Such protecting groups can be removed at a later stage of the synthesis using standard methods described in the literature. Examples of protecting groups are tert-butoxycarbonyl (Boc), 9-fluorenylmethyl carbamate (Fmoc), 2-trimethylsilylethyl carbamate (Teoc), carbobenzyloxy (Cbz) and p-methoxybenzyloxycarbonyl (Moz).

The compound of formula (I) can contain several asymmetric centers and can be present in the form of optically pure enantiomers, mixtures of enantiomers such as, for example, racemates, mixtures of diastereoisomers, diastereoisomeric racemates or mixtures of diastereoisomeric racemates.

The term “asymmetric carbon atom” means a carbon atom with four different substituents. According to the Cahn-Ingold-Prelog Convention an asymmetric carbon atom can be of the “R” or “S” configuration.

The invention thus relates to:

A compound according to the invention wherein R² chlorine;

A compound according to the invention wherein R³ is triazolylalkyl, morpholinylalkyl or imidazolylalkyl;

A compound according to the invention wherein R² is triazolylmethyl, methyl(oxo-dihydropyrimidinyl) methyl, morpholinylethyl, morpholinylmethyl, phenylmethyl, imidazolylethyl, (dioxo-thiazinanyl)ethyl or phenylmethyl; and

A compound according to the invention wherein R³ is triazolylmethyl, morpholinylmethyl or imidazolylethyl;

A compound of formula (I) selected from
4-(2-(1H-1,2,4-Triazol-1-yl)acetamido)-2′-chloro-4′-methyl-[1,1′-biphenyl]-3-carboxylic acid;
2′-chloro-4′-methyl-4-(2-(2-methyl-6-oxo-1,6-dihydropyrimidin-5-yl)acetamido)-[1,1′-biphenyl]-3-carboxylic acid;
2′-chloro-4′-methyl-4-(3-morpholinopropanamido)-[1,1′-biphenyl]-3-carboxylic acid;
5-(2-chloro-4-methylphenyl)-2-[(2-morpholin-4-ylacetyl)amino]benzoic acid;
5-(2-chloro-4-methylphenyl)-2-[(2-phenylacetyl)amino]benzoic acid;
5-(2-chloro-4-methylphenyl)-2-(2-imidazol-1-ylethoxy)benzoic acid;
5-(2-chloro-4-methylphenyl)-2-[2-(1,1-dioxo-1,4-thiazinan-4-yl)ethoxy]benzoic acid; and
5-(2-chloro-4-methylphenyl)-2-phenylmethoxybenzoic acid;
or a pharmaceutically acceptable salt or ester thereof.

The invention further relates to a compound of formula (I) selected from
4-(2-(1H-1,2,4-triazol-1-yl)acetamido)-2″-chloro-4″-methyl-[1,1″-biphenyl]-3-carboxylic acid;
5-(2-chloro-4-methylphenyl)-2-[(2-morpholin-4-ylacetyl)amino]benzoic acid; and
5-(2-chloro-4-methylphenyl)-2-(2-imidazol-1-ylethoxy)benzoic acid;
or a pharmaceutically acceptable salt or ester thereof.

The synthesis of the compound of formula (I) can, for example, be accomplished according to the following scheme. Unless otherwise indicated, R¹—R³ have the meaning as defined above.

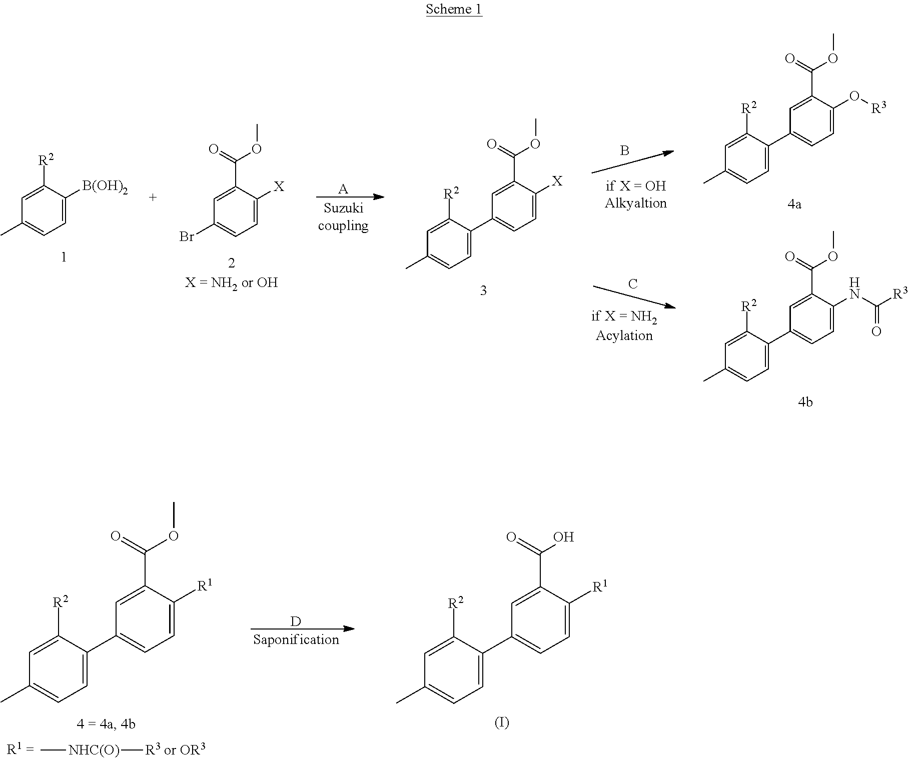

Step A: Coupling of the bromoderivative 2 with a suitable boronic acid or boronic acid ester 1 can be accomplished by using a palladium catalyst such as palladium(II)-acetate, palladium(II)-chloride, 1,1′-bis(diphenylphosphino)ferrocene-palladium (II)dichloride dichloromethane complex, tris(dibenzylideneacetone)dipalladium, tris(dibenzylideneacetone)dipalladium-chloroform adduct, or tetrakis(triphenylphosphine)palladium(0) in combination with a ligand such as triphenylphosphine, tricyclohexylphosphine, X-phos, Xantphos or the like, and a base such as potassium phosphate, potassium carbonate, cesium carbonate, triethylamine or diisopropylethylamine in a suitable solvent such as dioxane, toluene, dimethylacetamide, dimethylformamide, tetrahydrofuran, dimethoxyethane, diglyme, ethanol, methanol, water or mixtures of the solvents mentioned above at 20° C. to 180° C. for 5 min to 18 hrs with or without microwave irradiation.

Convenient conditions are the use of tris(dibenzylideneacetone)dipalladium-chloroform adduct, X-phos and potassium phosphate in a mixture of dioxane and water at 110° C. for 2 h.

Step B: Alkylation of the phenol 3 (X═OH) can be accomplished by reaction with a suitable alkylating agent such as substituted alkyl chlorides, substituted alkyl bromides, substituted alkyl iodides, substituted alkyl tosylates and a base such as cesium carbonate, potassium carbonate, sodium carbonate, triethylamine or ethyldiisopropylamine in a solvent such as as dioxane, dimethylacetamide, dimethylformamide, tetrahydrofuran at 0° C.-150° C. for 1 h to 18 h. Furthermore alkylation can be achieved by reaction of the phenol 3 (X═OH) with an alkohol R—OH in presence of an azodicarboxylate such as diethyl azodicarboxylate, diisopropyl azodicarboxylate or di-tert-butyl azodicarboxylate in presence of a phosphine such as triphenylphosphine, tricyclohexylphosphine or the like in a suitable solvent such as tetrahydrofuran, dichloromethane, chloroform, dimethylformamid, dimethylsulfoxide or acetonitrile at 0° C.-70° C. for 1 h-24 h.

Convenient conditions are the use of triphenylphosphine and di-tert-butyl azodicarboxylate in tetrahydrofuran at 25° C. for 18 h.

Step C: Acylation can be accomplished by a coupling reaction between amine 3 (X═NH₂) and carboxylic acids in the presence of a coupling reagent such as DCC, EDC, TBTU or HATU in the presence of an organic base such as triethylamine, diisopropylethylamine or N-methylmorpholine in various solvents such as dichloromethane, 1,2-dichloroethane, diethyl ether, dioxane, tetrahydrofuran or tert.-butyl methyl ether. Alternatively, amide formation can be accomplished by a coupling reaction between amine 3 (X═NH₂) and acyl chlorides in the presence of a base such as triethylamine, diisopropylethylamine or N-methylmorpholine in solvents such as dichloromethane, 1,2-dichloroethane, diethyl ether, dioxane, tetrahydrofuran or tert.-butyl methyl ether. Furthermore it can be accomplished by reaction of amine 3 (X═NH₂) with the carboxylic acid in presence of phosphorus oxychloride in pyridine. The before mentioned reactions can be performed at 0° C.-70° C. for 1-24 h.

Convenient conditions are the reaction of the acid with phosphorus oxychloride in pyridine at 25° C. for 1 h.

Step D: Saponification can be accomplished by reaction of the methyl ester 5 with a base such as lithium hydroxide, sodium hydroxide, potassium hydroxide or the like in a suitable solvent such as water, tetrahydrofuran, ethanol, methanol or mixtures thereof for 1-18 h at 0° C. to 90° C.

Advantageous conditions are the use of lithium hydroxide in a mixture of tetrahydrofuran and water at 65° C. for 4 h.

The invention thus also relates to a process for the preparation of a compound according to the invention comprising the saponification of a compound of formula (A1)

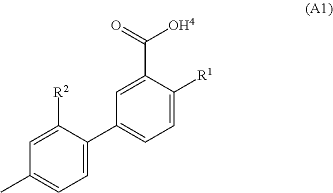

with a base or an acid, wherein R¹, R² and R³ are as defined above and R⁴ is alkyl.

R⁴ is advantageously methyl.

The base is conveniently lithium hydroxide, sodium hydroxide or potassium hydroxide, in particular lithium hydroxide.

A mixture of tetrahydrofuran and water is a convenient solvent for the process of the invention.

Preferred conditions for the process of the invention are the use of lithium hydroxide as a base in a mixture of tetrahydrofuran and water, at between around 0° C. to around 90° C., in particular at around 65° C. for 1 h to 18 h, in particular for 4 h.

The invention also relates to a compound according to the invention when manufactured according to a process of the invention.

Another embodiment of the invention provides a pharmaceutical composition or medicament containing a compound of the invention and a therapeutically inert carrier, diluent or excipient, as well as a method of using the compounds of the invention to prepare such composition and medicament. In one example, the compound of formula (I) may be formulated by mixing at ambient temperature at the appropriate pH, and at the desired degree of purity, with physiologically acceptable carriers, i.e., carriers that are non-toxic to recipients at the dosages and concentrations employed into a galenical administration form. The pH of the formulation depends mainly on the particular use and the concentration of compound, but preferably ranges anywhere from about 3 to about 8. In one example, a compound of formula (I) is formulated in an acetate buffer, at pH 5. In another embodiment, the compound of formula (I) is sterile. The compound may be stored, for example, as a solid or amorphous composition, as a lyophilized formulation or as an aqueous solution.

Compositions are formulated, dosed, and administered in a fashion consistent with good medical practice. Factors for consideration in this context include the particular disorder being treated, the particular mammal being treated, the clinical condition of the individual patient, the cause of the disorder, the site of delivery of the agent, the method of administration, the scheduling of administration, and other factors known to medical practitioners.

The compounds of the invention may be administered by any suitable means, including oral, topical (including buccal and sublingual), rectal, vaginal, transdermal, parenteral, subcutaneous, intraperitoneal, intrapulmonary, intradermal, intrathecal and epidural and intranasal, and, if desired for local treatment, intralesional administration. Parenteral infusions include intramuscular, intravenous, intraarterial, intraperitoneal, or subcutaneous administration.

The compounds of the present invention may be administered in any convenient administrative form, e.g., tablets, powders, capsules, solutions, dispersions, suspensions, syrups, sprays, suppositories, gels, emulsions, patches, etc. Such compositions may contain components conventional in pharmaceutical preparations, e.g., diluents, carriers, pH modifiers, sweeteners, bulking agents, and further active agents.

A typical formulation is prepared by mixing a compound of the present invention and a carrier or excipient. Suitable carriers and excipients are well known to those skilled in the art and are described in detail in, e.g., Ansel, Howard C., et al., Ansel's Pharmaceutical Dosage Forms and Drug Delivery Systems. Philadelphia: Lippincott, Williams & Wilkins, 2004; Gennaro, Alfonso R., et al. Remington: The Science and Practice of Pharmacy. Philadelphia: Lippincott, Williams & Wilkins, 2000; and Rowe, Raymond C. Handbook of Pharmaceutical Excipients. Chicago, Pharmaceutical Press, 2005. The formulations may also include one or more buffers, stabilizing agents, surfactants, wetting agents, lubricating agents, emulsifiers, suspending agents, preservatives, antioxidants, opaquing agents, glidants, processing aids, colorants, sweeteners, perfuming agents, flavoring agents, diluents and other known additives to provide an elegant presentation of the drug (i.e., a compound of the present invention or pharmaceutical composition thereof) or aid in the manufacturing of the pharmaceutical product (i.e., medicament).

The invention also relates in particular to:

A compound of formula (I) for use in the treatment of a disease modulated by cGAS;

The use of a compound of formula (I) for the treatment or prophylaxis of systemic lupus erythrematosus (SLE), cutaneous skin diseases like dermatomyositis or cutaneous lupus, interstitial pulmonary fibrosis, Sjogren syndrome, type I diabetes, inflammatory bowel disease, non-alcoholic steatohepatitis (NASH), juvenile inflammatory arthritis, ankylosing spondylitis, gout or Aicardi-Goutieres syndrome (AGS);

The use of a compound of formula (I) for the preparation of a medicament for the treatment or prophylaxis of systemic lupus erythrematosus (SLE), cutaneous skin diseases like dermatomyositis or cutaneous lupus, interstitial pulmonary fibrosis, Sjogren syndrome, type I diabetes, inflammatory bowel disease, non-alcoholic steatohepatitis (NASH), juvenile inflammatory arthritis, ankylosing spondylitis, gout or Aicardi-Goutieres syndrome (AGS);

A compound of formula (I) for use in the treatment or prophylaxis of systemic lupus erythrematosus (SLE), cutaneous skin diseases like dermatomyositis or cutaneous lupus, interstitial pulmonary fibrosis, Sjogren syndrome, type I diabetes, inflammatory bowel disease, non-alcoholic steatohepatitis (NASH), juvenile inflammatory arthritis, ankylosing spondylitis, gout or Aicardi-Goutieres syndrome (AGS); and

A method for the treatment or prophylaxis of systemic lupus erythrematosus (SLE), cutaneous skin diseases like dermatomyositis or cutaneous lupus, interstitial pulmonary fibrosis, Sjogren syndrome, type I diabetes, inflammatory bowel disease, non-alcoholic steatohepatitis (NASH), juvenile inflammatory arthritis, ankylosing spondylitis, gout or Aicardi-Goutieres syndrome (AGS), which method comprises administering an effective amount of a compound of formula (I) to a patient in need thereof.

The invention will now be illustrated by the following examples which have no limiting character.

### EXAMPLES

Abbreviations

DCM=dichloromethane; DMF=dimethylformamide; DMSO=dimethyl sulfoxide; ESI=electrospray ionization; EtOAc=ethyl acetate; HPLC=high performance liquid chromatography; MS=mass spectrometry; RT=room temperature.

### Synthesis of Intermediate A

### Methyl 4-amino-2′-chloro-4′-methyl-[1,1′-biphenyl]-3-carboxylate

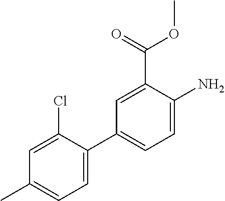 [SMILES: COC(=O)c1cc(-c2ccc(C)cc2Cl)ccc1N]

Methyl 2-amino-5-bromobenzoate (5 g, 21.7 mmol, Eq: 1), (2-chloro-4-methylphenyl) boronic acid (3.7 g, 21.7 mmol, Eq: 1) and potassium phosphate (9.23 g, 43.5 mmol, Eq: 2) were combined with dioxane (80 ml) and water (20 ml). The vial was degassed with argon before X-phos (518 mg, 1.09 mmol, Eq: 0.05) and tris(dibenzylideneacetone)dipalladium-chloroform adduct (562 mg, 543 μmol, Eq: 0.025) were added. The vial was closed and the reaction mixture was heated to 110° C. and stirred for 2 h. The reaction mixture was poured into 50 ml of water and extracted with EtOAc (3×50 ml). The organic layers were combined, dried over Na₂SO₄, filtered through sintered glass, concentrated and dried in vacuo. The crude material was purified by flash chromatography (silica gel, 120 g, 0% to 40% EtOAc in heptane). The fractions were combined, concentrated and dried in vacuo to afford the title compound methyl 4-amino-2′-chloro-4′-methyl-[1,1′-biphenyl]-3-carboxylate (5.08 g, 15.7 mmol, 72.3% yield) as brown solid. MS (ESI): 276.17 [M+H]+.

### Synthesis of Intermediate B

### Methyl 2′-chloro-4-hydroxy-4′-methyl-[1,1′-biphenyl]-3-carboxylate

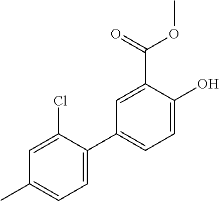 [SMILES: COC(=O)c1cc(-c2ccc(C)cc2Cl)ccc1O]

To a light yellow solution of methyl 5-bromo-2-hydroxybenzoate (2 g, 8.66 mmol, Eq: 1) in dioxane (40 ml) was added (2-chloro-4-methylphenyl)boronic acid (1.48 g, 8.66 mmol, Eq: 1). Potassium phosphate (3.67 g, 17.3 mmol, Eq: 2) solved in water (10 ml) was added. The reaction mixture was degassed during 2 min before X-phos (206 mg, 433 μmol, Eq: 0.05) and tris(dibenzylideneacetone)dipalladium-chloroform adduct (224 mg, 216 μmol, Eq: 0.025) were added. The mixture was heated to 100° C. for 1 hour. The reaction mixture was poured into 100 ml of water and extracted with EtOAc (3×100 ml). The organic layers were dried over MgSO₄ and concentrated in vacuo. The crude material was purified by flash chromatography (silica gel, 40 g, 0% to 20% EtOAc in heptane) to afford the title compound methyl 2′-chloro-4-hydroxy-4′-methyl-[1,1′-biphenyl]-3-carboxylate (1.9 g, 6.39 mmol, 73.8% yield) as light yellow solid, MS (ESI): 277.22 [M+H]+.

### Example 1

### 4-(2-(1H-1,2,4-Triazol-1-yl)acetamido)-2′-chloro-4′-methyl-[1,1′-biphenyl]-3-carboxylic Acid

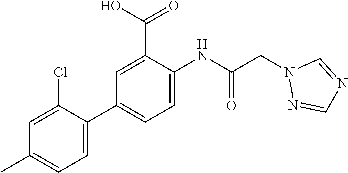 [SMILES: Cc1ccc(-c2ccc(NC(=O)Cn3cncn3)c(C(=O)O)c2)c(Cl)c1]

a) Methyl 4-(2-(1H-1,2,4-triazol-1-yl)acetamido)-2′-chloro-4′-methyl-[1,1′-biphenyl]-3-carboxylate

In a 5 ml vial, methyl 4-amino-2′-chloro-4′-methyl-[1,1′-biphenyl]-3-carboxylate, Intermediate A (80 mg, 290 μmol, Eq: 1) and 2-(1H-1,2,4-triazol-1-yl)acetic acid (36.9 mg, 290 μmol, Eq: 1) were combined with pyridine (1.25 ml). At −15° C. phosphorus oxychloride (55.2 mg, 33.5 μl, 360 μmol, Eq: 1.24) was added and the reaction mixture was stirred for 1 h. The crude reaction mixture was quenched with water, basified with 10 ml sat NaHCO₃ and extracted with EtOAc (4×50 ml). The crude reaction mixture was concentrated in vacuo. The crude material was purified by preparative HPLC to afford the title compound methyl 4-(2-(1H-1,2,4-triazol-1-yl)acetamido)-2′-chloro-4′-methyl-[1,1′-biphenyl]-3-carboxylate (76.8 mg, 197 μmol, 68% yield) as white solid, MS (ESI): 385.14 [M+H]+.

b) 4-(2-(1H-1,2,4-Triazol-1-yl)acetamido)-2′-chloro-4′-methyl-[1,1′-biphenyl]-3-carboxylic acid

To a light yellow solution (under argon in a 10 ml flask) of methyl 4-(2-(1H-1,2,4-triazol-1-yl) acetamido)-2′-chloro-4′-methyl-[1,1′-biphenyl]-3-carboxylate (40 mg, 104 μmol, Eq: 1) in tetrahydrofuran (1.2 ml) was added lithium hydroxide monohydrate (8.72 mg, 208 μmol, Eq: 2) solved in water (300 μl). The reaction mixture was heated to 65° C. and stirred during 4 hours. The mixture was quenched with HCl 2M (104 μl, 208 μmol, Eq: 2) and concentrated in vacuo. The crude material was triturated with diethyl ether (2×5 ml) and purified by preparative HPLC to obtain the title compound 4-(2-(1H-1,2,4-triazol-1-yl) acetamido)-2′-chloro-4′-methyl-[1,1′-biphenyl]-3-carboxylic acid (14.6 mg, 39.3 μmol, 37.8% yield) as white solid, MS (ESI): 371.21 [M+H]+.

### Example 2

### 2′-Chloro-4′-methyl-4-(2-(2-methyl-6-oxo-1,6-dihydropyrimidin-5-yl)acetamido)-[1,1′-biphenyl]-3-carboxylic Acid

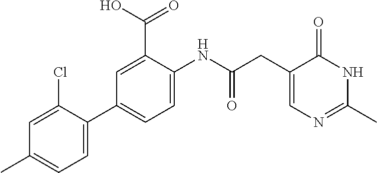

The title compound was obtained in comparable yield analogous to the procedure described for Example 1 using 2-(2-methyl-6-oxo-1,6-dihydropyrimidin-5-yl)acetic acid hydrochloride instead of 2-(1H-1,2,4-triazol-1-yl)acetic acid in step a), white solid, (MS (ESI): 412.22 [M+H]+.

### Example 3

### 2′-Chloro-4′-methyl-4-(3-morpholinopropanamido)-[1,1′-biphenyl]-3-carboxylic Acid

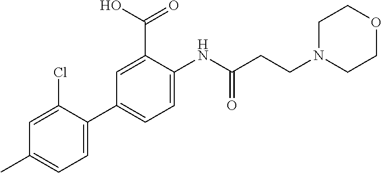 [SMILES: Cc1ccc(-c2ccc(NC(=O)CCN3CCOCC3)c(C(=O)O)c2)c(Cl)c1]

The title compound was obtained in comparable yield analogous to the procedure described for Example 1 using 3-morpholinopropanoic acid hydrochloride instead of 2-(1H-1,2,4-triazol-1-yl)acetic acid in step a), white solid, (MS (ESI): 403.26 [M+H]+.

### Example 4

### 5-(2-Chloro-4-methylphenyl)-2-[(2-morpholin-4-ylacetyl)amino]benzoic Acid

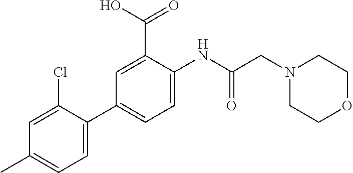 [SMILES: Cc1ccc(-c2ccc(NC(=O)CN3CCOCC3)c(C(=O)O)c2)c(Cl)c1]

The title compound was obtained in comparable yield analogous to the procedure described for Example 1 using 2-morpholinoacetic acid instead of 2-(1H-1,2,4-triazol-1-yl) acetic acid in step a), off-white solid, MS (ESI): 389.23 [M+H]+.

### Example 5

### 5-(2-Chloro-4-methylphenyl)-2-[(2-phenylacetyl)amino]benzoic Acid

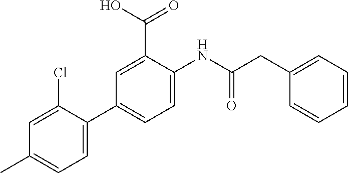 [SMILES: Cc1ccc(-c2ccc(NC(=O)Cc3ccccc3)c(C(=O)O)c2)c(Cl)c1]

a) Methyl 2′-chloro-4′-methyl-4-(2-phenylacetamido)-[1,1′-biphenyl]-3-carboxylate

To a light yellow solution of methyl 4-amino-2′-chloro-4′-methyl-[1,1′-biphenyl]-3-carboxylate, Intermediate A (75 mg, 272 μmol, Eq: 1) and triethylamine (55 mg, 75.8 μl, 544 μmol, Eq: 2) in dichloromethane (2 ml) was added 2-phenylacetyl chloride (92.5 mg, 79 μl, 598 μmol, Eq: 2.2). The reaction mixture was stirred at room temp during 4 hours. The reaction mixture was poured into 25 ml saturated NaHCO₃ solution and extracted with dichloromethane (3×25 ml). The organic layers were dried over MgSO₄ and concentrated in vacuo. The crude material was purified by flash chromatography (silica gel, 12 g, 0% to 30% EtOAc in heptane) to afford the title compound methyl 2′-chloro-4′-methyl-4-(2-phenylacetamido)-[1,1′-biphenyl]-3-carboxylate (67.3 mg, 123 μmol, 45.1% yield) as colorless oil. MS (ESI): 394.11 [M+H]+.

b) 5-(2-Chloro-4-methylphenyl)-2-[(2-phenylacetyl)amino]benzoic acid

The title compound was obtained in comparable yield analogous to the procedure described for Example 1 using methyl 2′-chloro-4′-methyl-4-(2-phenylacetamido)-[1,1′-biphenyl]-3-carboxylate in step b), light brown solid, MS (ESI): 380.19 [M+H]+.

### Example 6

### 5-(2-Chloro-4-methylphenyl)-2-(2-imidazol-1-ylethoxy)benzoic Acid

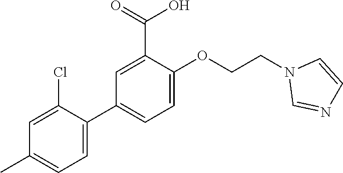 [SMILES: Cc1ccc(-c2ccc(OCCn3ccnc3)c(C(=O)O)c2)c(Cl)c1]

a) Methyl 4-(2-(1H-imidazol-1-yl)ethoxy)-2′-chloro-4′-methyl-[1,1′-biphenyl]-3-carboxylate

To a light yellow solution of methyl 2′-chloro-4-hydroxy-4′-methyl-[1,1′-biphenyl]-3-carboxylate, Intermediate B (100 mg, 361 μmol, Eq: 1), 2-(1H-imidazol-1-yl)ethan-1-ol (44.6 mg, 398 μmol, Eq: 1.1) and triphenylphosphine (114 mg, 434 μmol, Eq: 1.2) in tetrahydrofuran (2 ml) was added di-tert-butyl azodicarboxylate (108 mg, 470 μmol, Eq: 1.3). The reaction mixture was stirred at room temp over night. The reaction mixture was poured into 20 ml of water and extracted with EtOAc (3×20 ml). The organic layers were dried over MgSO₄ and concentrated in vacuo. The crude material was purified by flash chromatography (silica gel, 12 g, 0% to 5% MeOH in DCM) to afford the title compound methyl 4-(2-(1H-imidazol-1-yl)ethoxy)-2′-chloro-4′-methyl-[1,1′-biphenyl]-3-carboxylate (83.8 mg, 204 μmol, 56.5% yield) as colorless viscous oil. MS (ESI): 371.16 [M+H]+.

b) 5-(2-Chloro-4-methylphenyl)-2-(2-imidazol-1-ylethoxy)benzoic acid

The title compound was obtained in comparable yield analogous to the procedure described for Example 1 using methyl 4-(2-(1H-imidazol-1-yl)ethoxy)-2′-chloro-4′-methyl-[1,1′-biphenyl]-3-carboxylate in step b), white solid, MS (ESI): 357.19 [M+H]+.

### Example 7

### 5-(2-Chloro-4-methylphenyl)-2-[2-(1,1-dioxo-1,4-thiazinan-4-yl)ethoxy]benzoic Acid

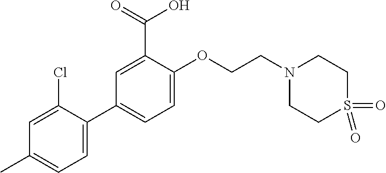 [SMILES: Cc1ccc(-c2ccc(OCCN3CCS(=O)(=O)CC3)c(C(=O)O)c2)c(Cl)c1]

The title compound was obtained in comparable yield analogous to the procedure described for Example 6 using 4-(2-hydroxyethyl)thiomorpholine 1,1-dioxide instead of 2-(1H-imidazol-1-yl)ethan-1-ol in step a), light brown solid, MS (ESI): 424.26 [M+H]+.

### Example 8

### 5-(2-Chloro-4-methylphenyl)-2-phenylmethoxybenzoic Acid

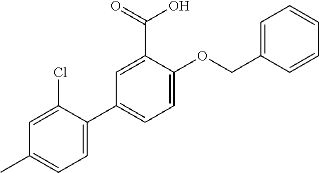 [SMILES: Cc1ccc(-c2ccc(OCc3ccccc3)c(C(=O)O)c2)c(Cl)c1]

a) Methyl 4-(benzyloxy)-2′-chloro-4′-methyl-[1,1′-biphenyl]-3-carboxylate

Methyl 2-(benzyloxy)-5-bromobenzoate (150 mg, 467 μmol, Eq: 1), (2-chloro-4-methylphenyl) boronic acid (79.6 mg, 467 μmol, Eq: 1) and potassium phosphate (tribasic) (198 mg, 934 μmol, Eq: 2) were combined with dioxane (2.5 ml) and water (625 μl). The vial was degassed with argon before X-phos (11.1 mg, 23.4 μmol, Eq: 0.05) and tris(dibenzylideneacetone)dipalladium-chloroform adduct (12.1 mg, 11.7 μmol, Eq: 0.025) were added. The vial was closed and the reaction mixture was heated to 110° C. and stirred for 2 h. The reaction mixture was poured into 40 ml of water and extracted with EtOAc (3×40 ml). The organic layers were combined, dried over MgSO₄, filtered through sintered glass, concentrated and dried in vacuo. The crude material was purified by flash chromatography (silica gel, 40 g, 0% to 40% EtOAc in heptane). The fractions were combined, concentrated and dried in vacuo. The compound was purified by preparative HPLC to afford the title compound methyl 4-(benzyloxy)-2′-chloro-4′-methyl-[1,1′-biphenyl]-3-carboxylate (46.4 mg, 126 μmol, 27.1% yield) as off-white solid, MS (ESI): 367.110 [M+H]+.

b) 5-(2-Chloro-4-methylphenyl)-2-phenylmethoxybenzoic acid

The title compound was obtained in comparable yield analogous to the procedure described for Example 1 using methyl 4-(benzyloxy)-2′-chloro-4′-methyl-[1,1′-biphenyl]-3-carboxylate in step b), off-white solid, MS (ESI): 353.14 [M+H]+.

### Example 9

### Malachite Green Assay to Measure cGAS Activity

Compounds were tested for cGAS inhibition in a coupled enzymatic assay based on Phosphate detection by Malachite Green. Final assay conditions were 20 mM TRIS pH 7.5 (Applichem), 5 mM MgCl₂ (Sigma) and 0.01% BSA (Sigma) supplemented with 80 μM ATP (Sigma), 80 μM GTP (Sigma) and 100 nM Interferon Stimulating DNA (ISD) (Microsynth). Recombinantly expressed purified human cGAS (residues 161-522) was used at 25 nM.

All compounds were prepared as 10 mM stock solutions in DMSO and a 16 pt dilution series in DMSO with a dilution factor of 2.5 was prepared. 1 μL of DMSO dilution series was transferred to 32.3 μL reaction buffer, mixed by pipetting up/down, spun for 1 minute at 3000 rpm and was visually inspected for precipitation. 5 μL of 3-fold enzyme stock solution were transferred to an empty 384-well Black/Clear Flat Bottom Polystyrene NBS (Corning) rows 3-24. Rows 1-2 were filled with assay buffer. Plates were spun 10 seconds at 1000 rpm (164×g). 5 μL of compound intermediate dilution was added and mixed by pipetting up/down to rows 3-24. Rows 1-2 were filled with 3.1% DMSO assay buffer. Plates were spun 10 seconds at 1000 rpm (164×g). 5 μL 3-fold Nucleotide/DNA mix was added to all wells to start the reaction. Plates were spun 10 seconds at 1000 rpm (164×g) and incubated for 4 hour at room temperature (RT) in the dark. 5 μL 4 U/mL PPase (Sigma) were added to all wells. Plates spun 10 seconds at 1000 rpm (164×g). 10 μL BioMol green Solution (Enzo Life Sciences) was added to all wells. Plates spun 10 seconds at 1000 rpm (164×g) and incubated 30 minutes at RT in the dark. Absorbance data was collected 620 nm on an EnVision Multilable Reader (Perkin Elmer) and the following measurement settings were used: excitation filter photometric was 620 nm; excitation from the top; measurement height was 1 mm; number of flashes was 30; number of flashes integrated was 1.

All plates are checked for abnormalities and outliers in the Blank Control (no protein, row 1) and the Neutral Control (no compound, row 2) are excluded using the 3*SD rule. Data was normalized to 0 and 100% by Blank and Neutral Control and each curve was fitted and judged using the 4 parameter logistic equation to determine the IC50 for cGAS inhibition.

The results of this assay are provided in Table 1. Table 1 provides IC50 values (μM) for cGAS inhibition obtained for particular examples of the present invention as measured by the above-described assay.

| Example | IC50 cGAS (μM) |
|---|---|
| 1 | 1.83 |
| 2 | 2.09 |
| 3 | 4.82 |
| 4 | 0.32 |
| 5 | 3.07 |
| 6 | 2.8 |
| 7 | 2.67 |
| 8 | 4.21 |

### Example A

Film coated tablets containing the following ingredients can be manufactured in a conventional manner:

| | |
|---|---|
| Ingredients | Per tablet |

| | | | | |
|---|---|---|---|---|
| Kernel: | | | | |
| Compound of formula (I) | 10.0 | mg | 200.0 | mg |
| Microcrystalline cellulose | 23.5 | mg | 43.5 | mg |
| Lactose hydrous | 60.0 | mg | 70.0 | mg |
| Povidone K30 | 12.5 | mg | 15.0 | mg |
| Sodium starch glycolate | 12.5 | mg | 17.0 | mg |
| Magnesium stearate | 1.5 | mg | 4.5 | mg |
| (Kernel Weight) | 120.0 | mg | 350.0 | mg |
| Film Coat: | | | | |
| Hydroxypropyl methyl cellulose | 3.5 | mg | 7.0 | mg |
| Polyethylene glycol 6000 | 0.8 | mg | 1.6 | mg |
| Talc | 1.3 | mg | 2.6 | mg |
| Iron oxide (yellow) | 0.8 | mg | 1.6 | mg |
| Titan dioxide | 0.8 | mg | 1.6 | mg |

The active ingredient is sieved and mixed with microcrystalline cellulose and the mixture is granulated with a solution of polyvinylpyrrolidone in water. The granulate is then mixed with sodium starch glycolate and magnesium stearate and compressed to yield kernels of 120 or 350 mg respectively. The kernels are lacquered with an aq. solution/suspension of the above mentioned film coat.

### Example B

Capsules containing the following ingredients can be manufactured in a conventional manner:

| Ingredients | Per capsule |
|---|---|
| Compound of formula (I) | 25.0 mg |
| Lactose | 150.0 mg |
| Maize starch | 20.0 mg |
| Talc | 5.0 mg |

The components are sieved and mixed and filled into capsules of size 2.

### Example C

Injection solutions can have the following composition:

| | |
|---|---|
| Compound of formula (I) | 3.0 mg |
| Polyethylene glycol 400 | 150.0 mg |
| Acetic acid | q.s. ad pH 5.0 |
| Water for injection solutions | ad 1.0 ml |

The active ingredient is dissolved in a mixture of Polyethylene glycol 400 and water for injection (part). The pH is adjusted to 5.0 by addition of acetic acid. The volume is adjusted to 1.0 ml by addition of the residual amount of water. The solution is filtered, filled into vials using an appropriate overage and sterilized.

1. A compound of formula (I) 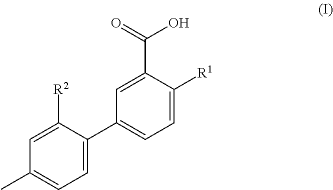
wherein
R¹ is —NHC(O)—R³ or —OR³;
R² is halogen; and
R³ is triazolylalkyl, alkyl(oxo-dihydropyrimidinyl)alkyl, morpholinylalkyl, imidazolylalkyl, (dioxo-thiazinanyl)alkyl or phenylalkyl;
or a pharmaceutically acceptable salt or ester thereof.

2. A compound according to claim 1, wherein R² chlorine.

3. A compound according to claim 1 or 2, wherein R³ is triazolylalkyl, morpholinylalkyl or imidazolylalkyl.

4. A compound according to any one of claims 1 to 3, wherein R³ is triazolylmethyl, morpholinylmethyl or imidazolylethyl.

5. A compound according to any one of claims 1 to 4 selected from
4-(2-(1H-1,2,4-triazol-1-yl)acetamido)-2′-chloro-4′-methyl-[1,1′-biphenyl]-3-carboxylic acid;
2′-chloro-4′-methyl-4-(2-(2-methyl-6-oxo-1,6-dihydropyrimidin-5-yl)acetamido)-[1,1′-biphenyl]-3-carboxylic acid;
2′-chloro-4′-methyl-4-(3-morpholinopropanamido)-[1,1′-biphenyl]-3-carboxylic acid;
5-(2-chloro-4-methylphenyl)-2-[(2-morpholin-4-ylacetyl)amino]benzoic acid;
5-(2-chloro-4-methylphenyl)-2-[(2-phenylacetyl)amino]benzoic acid;
5-(2-chloro-4-methylphenyl)-2-(2-imidazol-1-ylethoxy)benzoic acid;
5-(2-chloro-4-methylphenyl)-2-[2-(1,1-dioxo-1,4-thiazinan-4-yl)ethoxy]benzoic acid; and
5-(2-chloro-4-methylphenyl)-2-phenylmethoxybenzoic acid;
or a pharmaceutically acceptable salt or ester thereof.

6. A compound according to any one of claims 1 to 5 selected from
4-(2-(1H-1,2,4-triazol-1-yl)acetamido)-2′-chloro-4′-methyl-[1,1′-biphenyl]-3-carboxylic acid;
5-(2-chloro-4-methylphenyl)-2-[(2-morpholin-4-ylacetyl)amino]benzoic acid; and
5-(2-chloro-4-methylphenyl)-2-(2-imidazol-1-ylethoxy)benzoic acid;
or a pharmaceutically acceptable salt or ester thereof.

7. A process for the preparation of a compound according to any one of claims 1 to 6, comprising the following step:
the saponification of a compound of formula (A1) 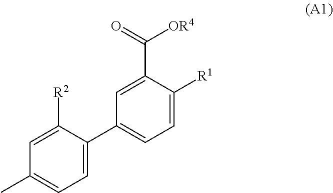
with a base or an acid in a suitable solvent for 1-18 h at 0°-70° C., wherein R¹, R² and R³ are as defined in any one of claims 1 to 7 and R⁴ is alkyl (C1-C8).

8. A compound according to any one of claims 1 to 6, when manufactured according to a process of claim 7.

9. A compound according to any one of claims 1 to 6 for use as therapeutically active substance.

10. A pharmaceutical composition comprising a compound in accordance with any one of claims 1 to 6 and a therapeutically inert carrier.

11. The use of a compound according to any one of claims 1 to 6 for the treatment or prophylaxis of systemic lupus erythrematosus (SLE), cutaneous skin diseases like dermatomyositis or cutaneous lupus, interstitial pulmonary fibrosis, Sjogren syndrome, type I diabetes, inflammatory bowel disease, non-alcoholic steatohepatitis (NASH), juvenile inflammatory arthritis, ankylosing spondylitis, gout or Aicardi-Goutieres syndrome (AGS).

12. The use of a compound according to any one of claims 1 to 6 for the preparation of a medicament for the treatment or prophylaxis of systemic lupus erythrematosus (SLE), cutaneous skin diseases like dermatomyositis or cutaneous lupus, interstitial pulmonary fibrosis, Sjogren syndrome, type I diabetes, inflammatory bowel disease, non-alcoholic steatohepatitis (NASH), juvenile inflammatory arthritis, ankylosing spondylitis, gout or Aicardi-Goutieres syndrome (AGS).

13. A compound according to any one of claims 1 to 6 for use in the treatment or prophylaxis of systemic lupus erythrematosus (SLE), cutaneous skin diseases like dermatomyositis or cutaneous lupus, interstitial pulmonary fibrosis, Sjogren syndrome, type I diabetes, inflammatory bowel disease, non-alcoholic steatohepatitis (NASH), juvenile inflammatory arthritis, ankylosing spondylitis, gout or Aicardi-Goutieres syndrome (AGS).

14. A method for the treatment or prophylaxis of systemic lupus erythrematosus (SLE), cutaneous skin diseases like dermatomyositis or cutaneous lupus, interstitial pulmonary fibrosis, Sjogren syndrome, type I diabetes, inflammatory bowel disease, non-alcoholic steatohepatitis (NASH), juvenile inflammatory arthritis, ankylosing spondylitis, gout or Aicardi-Goutieres syndrome (AGS), which method comprises administering an effective amount of a compound as defined in any one of claims 1 to 6 to a patient in need thereof.

15. The invention as hereinbefore described.
